(19) United States
(12) Patent Application Publication
(10) Pub. No.: US 2021/0228763 A1
(43) Pub. Date: Jul. 29, 2021

(54) SYSTEM AND METHOD FOR SYNTHESIZING GRAPHENE SUPPORTED PHOTOCATALYTIC NANOMATERIALS FOR AIR PURIFICATION

(71) Applicant: LOG 9 MATERIALS SCIENTIFIC PRIVATE LIMITED, BENGALURU (IN)

(72) Inventors: AKSHAY VIVEK SINGHAL, DEOBAND (IN); ANSHUL KUMAR SHARMA, PALAMPUR (IN); KUNAL PAUL, KOLKATA (IN); SYED SHAJAR ALI IMAM, NAMRUP (IN)

(21) Appl. No.: 17/259,546

(22) PCT Filed: Jul. 9, 2019
(86) PCT No.: PCT/IN2019/050506

§ 371 Date: Jan. 11, 2021

(30) Foreign Application Priority Data
Jul. 9, 2018 (IN) 201811000975

(51) Int. Cl. A61L 9/20; B01J 21/06; B01J 23/06; B01J 23/14; B01J 21/18; B01J 21/04; B01J 21/08; B01J 35/00; B01J 35/02; B01J 37/02; B01J 37/06; B01J 37/00; B01J 37/04; B01J 37/08; B01J 37/34

(52) U.S. Cl. CPC A61L 9/205; A61L 2209/15; B01J 23/06; B01J 23/14; B01J 21/18; B01J 21/04; B01J 21/08; B01J 21/066; B01J 35/004; B01J 35/026; B01J 37/0244; B01J 37/0228; B01J 37/0219; B01J 37/06; B01J 37/009; B01J 37/04; B01J 37/084; B01J 37/088; B01J 37/345; A61L 2209/12; A61L 2209/14; B01J 21/063

(57) ABSTRACT

The embodiments herein provide a system and a method for synthesizing graphene-supported photocatalytic nanomaterials for air purification. The method includes synthesizing a ceramic substrate from a ceramic material in particulate form; depositing carbon material on the synthesized ceramic substrate; depositing one photocatalytic nanomaterial on the carbonaceous material coated ceramic substrate; transforming the phase of the ceramic substrate coated with carbonaceous photocatalytic nanomaterial in inert atmospheric condition from one phase to another phase; and activating the transformed ceramic substrate coated with carbonaceous photocatalytic nanomaterial, when exposed to photo energy source.

### CROSS-REFERENCE TO RELATED APPLICATIONS

The present application is a National Phase Application of the PCT application with the serial number PCT/IN2019/050506 filed on Jul. 9, 2019 with the title, “SYSTEM AND METHOD FOR SYNTHESIZING GRAPHENE SUPPORTED PHOTOCATALYTIC NANOMATERIALS FOR AIR PURIFICATION”. The present application claims the priority of the Indian Provisional Patent Application (PPA) with serial number 201811000975 filed on Jan. 9, 2018 and subsequently postdated by 6 Months to Jul. 9, 2018 with the title, “GRAPHENE SUPPORTED PHOTOCTALYST NANOMATERIALS FOR AIR PURIFICATION”, the contents of abovementioned Provisional Patent application and PCT applications are included in entirety as reference herein.

### BACKGROUND

### Technical Field

The embodiments herein are generally related to a field of air purification systems and methods. The embodiments herein are particularly related to photocatalyst nanomaterials for air purification. The embodiments herein are more particularly related to graphene supported photocatalyst nanomaterials, for efficiently removing a plurality of toxic gaseous pollutants such as NOx, SOx, VOCs and other organic pollutants present in air.

### Description of the Related Art

Indoor air quality is of great importance for human health. The human population spends most of their time in the houses, offices and cars. For example, formaldehyde (HCHO) is considered as one of the foremost and injurious indoor volatile organic compounds (VOCs). These indoor pollutants are considered harmful to humans as they cause irritation to respiratory and sensory system. The long-term exposure to formaldehyde at concentrations as low as 0.03 ppm can lead to tears, breathing problems and other symptoms such as headache and nausea.

One of the chief pollutants in the atmosphere is oxides of nitrogen (NOx such as NO and NO₂). The main source of NOx in the air mainly comes from exhaust of vehicles, fossil fuel combustion and emissions from stationary sources. The nitrogen oxides released into the air, mix with other chemicals present in the air to form acid rain, and photochemical smog. The acid rain and photochemical smog cause damage to human health.

Sulphur dioxide (SO₂) is another major pollutant with impact on both environment as well as human health. The industrial activities that burn sulphur related fossil fuels and motor vehicle emissions release sulfur dioxide to the environment. These pollutants cause respiratory problems and eye irritations. Hence removal of sulphur dioxide (SO₂) present in the air is very important.

Currently available air purifiers in the market are only efficient in removal of particulate matter (PM). The currently available air purifiers do not exhibit catalytic activity for removing the pollutants such as NOx, SOx and volatile organic compounds which are the major pollutants in the air. The deodorization filter available in the market has problems such as poor performance and short lifetime. Further the deodorization filter is not able to treat the harmful microbes in the air.

To resolve the above problems, there exists a need for photocatalyst technologies with strong adsorption capacity. The photocatalyst nanomaterials form different radicals after excitation by an exposure to a photo-energy source.

It is desirable to use photocatalyst nanomaterials that forms different radicals for providing strong catalytic activity and oxidizing power to sterilize microbes and to decompose volatile organic substances which cause odor.

Further, it is desirable to use graphene derivatives supported photocatalyst nanoparticles with surface-active of groups for promoting photo-oxidation and adsorption of plurality of pollutant gases simultaneously. The plurality of pollutant gases is adsorbed through electron transfer ability of graphene and photocatalytic properties of oxides of Titanium (Ti), Zinc (Zn), Tin (Sn) and the like.

Furthermore, it is desirable to have graphene derivatives supported photocatalyst nanomaterials that remove pollutants such as NOx, SOx and volatile organic compounds (VOCs) by adsorption, absorption or catalytic conversion with high efficiency. Advance oxidation process and photocatalytic reduction of all harmful gases take place in presence of photo energy source.

Hence, there is a need to provide a method and an air purification system for cleaning and improving the indoor air quality. In addition, there is a need to provide an air purification system for eliminating allergens, unpleasant smells and major pollutants in the air.

Further, there is a need to provide air filters for the simultaneous removal of major pollutants including HCHO, NOx and SOx and other organic pollutants.

The above shortcomings, disadvantages and problems are addressed herein, which will be understood by studying the following specifications.

### OBJECTIVES OF THE EMBODIMENTS

The primary objective of the embodiments herein is to provide a graphene based active material filter bed system for use in domestic and industrial applications for removing harmful toxic components present in air.

Another objective of the embodiments herein is to provide an active filter bed system comprising a photocatalytic nanomaterial coated strongly on a ceramic substrate for catalytic degradation of gaseous and volatile pollutants for purifying air.

Yet another objective of the embodiments herein is to provide a photo energy source comprising but not limited to Ultraviolet Light Source for activating photocatalytic material of the bed.

Yet another objective of the embodiments herein is to provide an Ultraviolet Light Source that also acts as a germicidal eliminator and effectively removes microorganisms like bacteria, viruses, yeasts and fungal spores.

Yet another objective of the embodiments herein is to provide a graphene based filter comprising active material containing graphene supported metal oxide nanoparticles of Titanium, Zinc or Tin, and like for air purification in a filtration bed.

Yet another objective of the embodiments herein is to provide a graphene based nanofiltration system comprising the graphene based active material coated strongly on ceramics substrate like alumina, silica, magnesia, zirconia, iron oxide, etc., for air purification.

Yet another objective of the embodiments herein is to provide an air purifier active material bed, filled with the active material either in granular form or sintered ceramic bed form, or rod-shaped form.

Yet another objective of the embodiments herein is to provide a graphene supported nanomaterials-based filter system for air purification, for simultaneously removing major gaseous pollutants such as NOx, SOx and volatile pollutants.

Yet another objective of the embodiments herein is to provide a graphene supported nanomaterials-based filter bed for air purification, by chemically functionalizing the graphene to remove volatile organic compounds (VOCs) including HCHO, benzene and the like.

Yet another objective of the present invention is to provide a graphene supported nanomaterial-based filter system for air purification, and for removing bad odor.

Yet another objective of the embodiments herein is to provide a graphene supported nanomaterials-based filter system endowed with antimicrobial activity for air purification.

These and other objects and advantages of the embodiments herein will become readily apparent from the following detailed description taken in conjunction with the accompanying drawings.

### SUMMARY

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The other objects and advantages of the embodiments herein will become readily apparent from the following description taken in conjunction with the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

According to one embodiment herein, a method of synthesizing graphene-supported photocatalytic nanomaterials for air purification is provided. The method comprises the following steps of synthesizing a ceramic substrate from a ceramic material in particulate form, and wherein the ceramic material is selected from a group consisting of silica, alumina, zirconia, and metal oxide; depositing carbonaceous material on the synthesized ceramic substrate to synthesize ceramic substrate coated with carbonaceous material, and wherein the carbonaceous material is selected from a group consisting of sugar, asphalt; depositing at least one photocatalytic nanomaterial on the ceramic substrate coated with carbonaceous material, wherein the at least one photocatalytic nanomaterial is selected from a group consisting of metal oxides of Titanium (Ti), Tin (Sn), and Zinc(Zn); transforming the phase of the ceramic substrate coated with carbonaceous photocatalytic nanomaterial in an inert atmospheric condition from one phase to another phase; and activating the transformed ceramic substrate coated with carbonaceous photocatalytic nanomaterial, upon exposure to a photo energy source.

According to embodiment herein, an air purification system is disclosed. The air purification system comprises a detachable air filter bed. The air filter bed further comprises a bed frame packed with a plurality of blocks. Each one of the plurality of blocks is configured for supporting and holding graphene supported photocatalytic nanomaterials, wherein the graphene supported photocatalytic nanomaterials are synthesized by synthesizing a ceramic substrate from a ceramic material in particulate form; depositing carbonaceous material on the synthesized ceramic substrate to obtain a ceramic substrate coated with carbonaceous material; depositing at least one photocatalytic nanomaterial on the ceramic substrate coated with carbonaceous material; transforming a phase of the ceramic substrate coated with carbonaceous photocatalytic nanomaterial in an inert atmospheric condition from one phase to another phase; and activating the transformed ceramic substrate coated with carbonaceous photocatalytic nanomaterial, upon exposure to photo energy source, and wherein the ceramic material is selected from a group consisting of silica, alumina, zirconia, and metal oxide;, wherein the carbon material is selected from a group consisting of sugar, asphalt; wherein the at least one photocatalytic nanomaterial is selected from a group consisting of metal oxides of Titanium (Ti), Tin (Sn), and Zinc (Zn).

According to one embodiment herein, the photo energy source comprises an ultraviolet source of light for activating the graphene supported photocatalytic material present in the filter bed.

According to one embodiment herein, a graphene based active material filter system is provided, and wherein the active material comprises graphene supported metal oxide nanoparticles of Titanium, Zinc or Tin and the like.

According to one embodiment herein, an active filter bed for air purification is provided to exhibit an enhanced photocatalytic activity upon the ultraviolet and visible light irradiation. The active filter bed material comprises graphene supported/doped metal oxide nanoparticles of Titanium, Zinc or Tin and the like.

According to one embodiment herein, a graphene based active material bed is provided for the air purification, and wherein the active materials are in a granular or rod shaped or sintered form.

According to one embodiment herein, a nano filtration media for air purification is provided, and wherein the nano-filtration media comprises metal oxide nanoparticles strongly adhered on the ceramic substrate. The metal oxide nanoparticles are selected from a group consisting of Titanium, Zinc or Tin, and the like. The ceramic substrate is selected from a group consisting of alumina, silica, magnesia, zirconia, iron oxide and the like.

According to one embodiment herein, a method of synthesizing graphene-supported photocatalytic nanomaterials for air purification is provided. The method comprises the following steps. A ceramic material is preprocessed. The pre-processing of the ceramic material comprises washing and drying of the ceramic material to obtain the ceramic material free from contaminants and surface activation. A carbonaceous material is synthesized and deposited on the preprocessed ceramic material to obtain a ceramic substrate coated with carbonaceous material. A catalytic material is deposited on the ceramic substrate coated with carbonaceous material by in-situ deposition of oxides of Ti, Zn, Sn and the like on the ceramic substrate coated with carbonaceous materials. The ceramic substrate coated with carbonaceous materials and deposited with the catalytic material is subjected to carbonization and phase transformation. The ceramic substrate coated with carbonaceous materials and deposited with the catalytic material is annealed in an inert atmosphere annealing for carbonization of carbonaceous material and phase transformation of hydroxide to oxide of Ti/Zn/Sn deposited on the particles coated with carbonaceous materials simultaneously. The ceramic substrate coated with carbonaceous materials and deposited with catalytic material is subjected to activation after completion of carbonization and phase transformation process. The step of activating the ceramic substrate coated with carbonaceous materials and deposited with catalytic material comprises an UV activation process and acid/base activation or other functionalization of the particles coated with carbonaceous materials. The activated ceramic substrate coated with carbonaceous materials and deposited with catalytic material is subjected to washing/neutralization process. The washed/neutralized ceramic substrate coated with carbonaceous materials and deposited with catalytic material is packing in frame.

According to one embodiment herein, an active material comprising a graphene supported nanomaterial for air purification is provided to eliminate gaseous pollutants such as NOx, SOx and toxic volatile pollutants. This active material is activated under a UV source provided within the system. The active material is also configured to act as germicidal eliminator and effectively remove microorganisms like bacteria, viruses, yeasts and fungal spores.

According to one embodiment herein, an active material comprising chemically functionalized graphene supported nanostructures for air purification is provided to efficiently remove the volatile organic compounds (VOCs) such as formaldehyde, benzene and the like. The graphene supported nanostructures filled in the filter bed are effective in removal of bad odor.

According to one embodiment herein, an active material filter comprises a graphene supported nanomaterial with effective antimicrobial and antibacterial property for air purification.

According to one embodiment herein, an active filter bed comprising graphene supported photocatalyst nanomaterials for air purification is provided. A synthesis of the graphene based photocatalyst on ceramic material comprises a preprocessing step in which the ceramic material is segregated based on desired size and shape. The ceramic material in particulate form is first washed with deionized water properly and dried by heating so that the ceramic material is free from contaminants (Step 1).

The step of washing and drying decontaminates the ceramic materials and activates the surface of the ceramic particles. After washing and drying, a carbonaceous material is deposited on the ceramic material by heating the ceramic material in a solution of carbon precursors to get a uniformly coated layer of carbonaceous material. The ceramic material is heated at a temperature range of 150-250° C. (Step 2).

After coating the ceramic substrate with a uniform layer of carbonaceous material, synthesis and deposition of catalytic materials onto the ceramic material is done. In this step, the photocatalytic materials such as metal oxides of Titanium (Ti), Zinc (Zn), Tin (Sn) and the like, are synthesized by the dropwise addition of the precursor into a suitable solvent in an appropriate quantity under continuous stirring. The pH of the solution is maintained by the adding an acid. Now with the addition of ceramic material into the mixture, the deposition process of metal oxides of Titanium (Ti), Zinc (Zn), Tin (Sn) and other active oxide nanoparticles is initiated. The hydrolysis of the precursor is carried out using a mixed solvent which is added in a dropwise manner. A thick sol-gel is formed to indicate the formation of metal hydroxides. The sol-gel mixture is allowed to be mixed properly in a magnetic stirrer (Step 3).

The ceramic substrate coated with layer of carbonaceous material and catalytic material is subjected to carbonization and the phase transformation process. In the carbonization and the phase transformation process, first the mixture prepared is subjected to slow heating to undergo phase transformation from a gel phase to a dry phase. The sol-gel mixture is then annealed at a very high temperature at a heating rate of 1-10° C./min up to a temperature of 850° C. in tubular furnace in an inert atmospheric condition. The annealing is carried out to achieve the carbonization of carbonaceous material and the photocatalytic material undergoes phase transformation from its hydroxide form to its oxide simultaneously (Step 4).

After the completion of carbonization and the phase transformation process, the ceramic substrate coated with layer of carbonaceous material and catalytic material is subjected to UV activation and acid/base activation (Step 5).

The ceramic substrate coated with layer of carbonaceous material and catalytic material is washed and neutralized, after the completion of UV activation and acid/base activation processes to synthesize the graphene supported photocatalyst nanomaterials comprises the (Step 6).

Once the graphene supported photocatalyst nanomaterials coated over ceramic is ready, the graphene supported photocatalyst nanomaterials coated over ceramic is filled in the plastic frame which acts as attachable-detachable type filter and wherein a design of filter frame is varied based on the application or requirements such as Indoor Air Purifier, Air Conditioner, Outdoor Industrial applications etc.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of illustration and not of limitation. Many changes and modifications may be made within the scope of the embodiments herein without departing from the spirit thereof, and the embodiments herein include all such modifications.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart explaining a method for synthesizing graphene based active material composite for air purification, according to one embodiment herein.

FIG. 2 illustrates a front side perspective view of attachable-detachable type of air filter bed material, according to one embodiment herein.

FIG. 3 illustrates a Scanning Electron Microscopy (SEM) images indicating the photocatalytic material coated on the surface of graphene supported ceramic material/ceramic substrate, according to one embodiment herein.

Although the specific features of the present invention are shown in some drawings and not in others. This is done for convenience only as each feature may be combined with any or all of the other features in accordance with the present invention.

### DETAILED DESCRIPTION OF THE EMBODIMENTS

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that other changes may be made without departing from the scope of the embodiments. The following detailed description is therefore not to be taken in a limiting sense.

The various embodiments herein provide a graphene based active material filter for domestic as well as industrial purposes, for absorbing harmful toxic gases and odors to give fresh and breathable air. Also, the embodiments herein provide an active filter bed system for purification of air. The active filter bed comprises a photocatalytic material for catalytic degradation of gaseous and volatile pollutants, wherein the photocatalytic material is coated strongly on a ceramic base/substrate/material.

According to an embodiment herein, a graphene based active material system for filtration of air is provided. The graphene based active material filter based system is a standalone product and is attached to any brand, class or grade of air filter products such as indoor air filters, industrial air filters, automobile air filters and also air-conditioning systems and the like.

According to one embodiment herein, a photocatalyst supported active filter material is provided, where the photocatalyst is coated on a ceramic material with integrated ultraviolet lamp grid for removal of toxic volatile components present in the air.

According to one embodiment herein, a photocatalyst based graphene-ceramic composite based material filter bed is fabricated. The photocatalyst ceramic composite is used in granular or sintered form for removal of pollutants present in the air. The graphene-based composite is chemically functionalized by addition of metal oxides of Titanium (Ti), Zinc (Zn), Tin (Sn) and other active oxides of nano-particles for removal of VOCs, NOx and SOx pollutants gases, dehumidification and removing bad odour from air.

According to one embodiment herein, an active material filter bed frame is provided. The design of filter frame varies according to the application or requirements. The frame holds the active material composite and allows effective flow of air through the filter bed which allows more air to pass through the active material.

According to one embodiment herein, a method is provided for economically viable synthesis of active material composite with cost effective precursor materials.

According to one embodiment herein, a method of synthesizing graphene-supported photocatalytic nanomaterials for air purification is provided. The method comprises the following steps of synthesizing a ceramic substrate from a ceramic material in particulate form, and wherein the ceramic material is selected from a group consisting of silica, alumina, zirconia, and metal oxide; depositing carbonaceous material on the synthesized ceramic substrate to synthesize ceramic substrate coated with carbonaceous material, and wherein the carbonaceous material is selected from a group consisting of sugar, asphalt; depositing at least one photocatalytic nanomaterial on the ceramic substrate coated with carbonaceous material, wherein the at least one photocatalytic nanomaterial is selected from a group consisting of metal oxides of Titanium (Ti), Tin (Sn), and Zinc (Zn); transforming the phase of the ceramic substrate coated with carbonaceous photocatalytic nanomaterial in an inert atmospheric condition from one phase to another phase; and activating the transformed ceramic substrate coated with carbonaceous photocatalytic nanomaterial, upon exposure to a photo energy source.

According to embodiment herein, an air purification system is disclosed. The air purification system comprises a detachable air filter bed. The air filter bed further comprises a bed frame packed with a plurality of blocks. Each one of the plurality of blocks is configured for supporting and holding graphene supported photocatalytic nanomaterials, wherein the graphene supported photocatalytic nanomaterials are synthesized by synthesizing a ceramic substrate from a ceramic material in particulate form; depositing carbonaceous material on the synthesized ceramic substrate to obtain a ceramic substrate coated with carbonaceous material; depositing at least one photocatalytic nanomaterial on the ceramic substrate coated with carbonaceous material; transforming a phase of the ceramic substrate coated with carbonaceous photocatalytic nanomaterial in an inert atmospheric condition from one phase to another phase; and activating the transformed ceramic substrate coated with carbonaceous photocatalytic nanomaterial, upon exposure to photo energy source, and wherein the ceramic material is selected from a group consisting of silica, alumina, zirconia, and metal oxide;, wherein the carbon material is selected from a group consisting of sugar, asphalt; wherein the at least one photocatalytic nanomaterial is selected from a group consisting of metal oxides of Titanium (Ti), Tin (Sn), and Zinc(Zn).

According to one embodiment herein, the photo energy source comprises an ultraviolet source of light for activating the graphene supported photocatalytic material present in the filter bed.

According to one embodiment herein, a graphene based active material filter system is provided, and wherein the active material comprises graphene supported metal oxide nanoparticles of Titanium, Zinc or Tin and the like.

According to one embodiment herein, an active filter bed for air purification is provided to exhibit an enhanced photocatalytic activity upon the ultraviolet and visible light irradiation. The active filter bed material comprises graphene supported/doped metal oxide nanoparticles of Titanium, Zinc or Tin and the like.

According to one embodiment herein, a graphene based active material bed is provided for the air purification, and wherein the active materials are in a granular or rod shaped or sintered form.

According to one embodiment herein, a nano filtration media for air purification is provided, and wherein the nano-filtration media comprises metal oxide nanoparticles strongly adhered on the ceramic substrate. The metal oxide nanoparticles are selected from a group consisting of Titanium, Zinc or Tin, and the like. The ceramic substrate is selected from a group consisting of alumina, silica, magnesia, zirconia, iron oxide and the like.

According to one embodiment herein, a method of synthesizing graphene-supported photocatalytic nanomaterials for air purification is provided. The method comprises the following steps. A ceramic material is preprocessed. The pre-processing of the ceramic material comprises washing and drying of the ceramic material to obtain the ceramic material with surface activation and free from contaminants. A carbonaceous material is synthesized and deposited on the preprocessed ceramic material to obtain a ceramic substrate coated with carbonaceous material. A catalytic material is deposited on the ceramic substrate coated with carbonaceous material by in-situ deposition of oxides of Ti, Zn, Sn and the like on the ceramic substrate coated with carbonaceous materials. The ceramic substrate coated with carbonaceous materials and deposited with the catalytic material is subjected to carbonization and phase transformation. The ceramic substrate coated with carbonaceous materials and deposited with the catalytic material is annealed in an inert atmosphere for carbonization of carbonaceous material and phase transformation of hydroxide to oxide of Ti/Zn/Sn deposited on the particles coated with carbonaceous materials simultaneously. The ceramic substrate coated with carbonaceous materials and deposited with catalytic material is subjected to activation after completion of carbonization and phase transformation process. The step of activating the ceramic substrate coated with carbonaceous materials and deposited with catalytic material comprises a UV activation process and acid/base activation or other functionalization of the particles coated with carbonaceous materials. The activated ceramic substrate coated with carbonaceous materials and deposited with catalytic material is subjected to washing/neutralization process. The washed/neutralized ceramic substrate coated with carbonaceous materials and deposited with catalytic material is packed in frame.

According to one embodiment herein, an active material comprising a graphene supported nanomaterial for air purification is provided to eliminate gaseous pollutants such as NOx, SOx and toxic volatile pollutants. This active material is activated under a UV source provided within the system. The active material is also configured to act as germicidal eliminator and effectively remove microorganisms like bacteria, vinises, yeasts and fungal spores.

According to one embodiment herein, an active material comprising chemically functionalized graphene supported nanostructures for air purification is provided to efficiently remove the volatile organic compounds (VOCs) such as formaldehyde, benzene and the like. The graphene supported nanostructures filled in the filter bed are effective in removal of bad odor.

According to one embodiment herein, an active material filter comprises a graphene supported nanomaterial with effective antimicrobial and antibacterial property for air purification.

According to one embodiment herein, an active filter bed comprising graphene supported photocatalyst nanomaterials for air purification is provided. A synthesis of the graphene based photocatalyst on ceramic material comprises a preprocessing step in which the ceramic material is segregated based on desired size and shape. The ceramic material in particulate form is first washed with deionized water properly and dried by heating so that the ceramic material is free from contaminants (Step 1).

The step of washing and drying decontaminates the ceramic materials and activates the surface of the ceramic particles. After washing and drying, a carbonaceous material is deposited on the ceramic material by heating the ceramic material in a solution of carbon precursors to get a uniformly coated layer of carbonaceous material. The ceramic material is heated at a temperature range of 150-250° C. (Step 2).

After coating the ceramic substrate with a uniform layer of carbonaceous material, synthesis and deposition of catalytic materials onto the ceramic material is done. In this step, the photocatalytic materials such as metal oxides of Titanium (Ti), Zinc (Zn), Tin (Sn) and the like, are synthesized by the dropwise addition of the precursor into a suitable solvent in an appropriate quantity under continuous stirring. The pH of the solution is maintained by the adding an acid. Now with the addition of ceramic material into the mixture, the deposition process of metal oxides of Titanium (Ti), Zinc (Zn), Tin (Sn) and other active oxide nanoparticles is initiated. The hydrolysis of the precursor is carried out using a mixed solvent which is added in a dropwise manner A thick sol-gel is formed to indicate the formation of metal hydroxides. The sol-gel mixture is allowed to be mixed properly in a magnetic stirrer (Step 3).

The ceramic substrate coated with layer of carbonaceous material and catalytic material is subjected to carbonization and the phase transformation process. In the carbonization and the phase transformation process, first the mixture prepared is subjected to slow heating to undergo phase transformation from a gel phase to a dry phase. The sol-gel mixture is then annealed at a very high temperature at a heating rate of 1-10° C./min up to a temperature of 850° C. in tubular furnace in an inert atmospheric condition. The annealing is carried out to achieve the carbonization of carbonaceous material and the photocatalytic material undergoes phase transformation from its hydroxide form to its oxide simultaneously (Step 4).

After the completion of carbonization and the phase transformation process, the ceramic substrate coated with layer of carbonaceous material and catalytic material is subjected to UV activation and acid/base activation (Step 5).

The ceramic substrate coated with layer of carbonaceous material and catalytic material is washed and neutralized, after the completion of UV activation and acid/base activation processes to synthesize the graphene supported photocatalyst nanomaterials comprises the washing for materials & neutralization (Step 6).

Once the graphene supported photocatalyst nanomaterials coated over ceramic is ready, the graphene supported photocatalyst nanomaterials coated over ceramic is filled in the plastic frame which acts as attachable-detachable type filter and wherein a design of filter frame is varied based on the application or requirements such as Indoor Air Purifier, Air Conditioner, Outdoor Industrial applications etc.

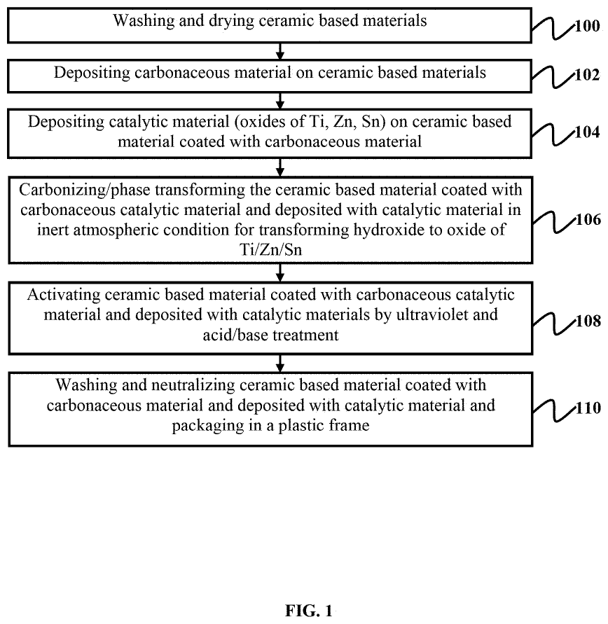

FIG. 1 is a flow chart illustrating a process for synthesizing graphene based active material composite for air purification, according to one embodiment herein. The ceramic material of desired size is obtained. The ceramic based material is washed and dried (Step 100). The ceramic based material is dried by heating so that it is free from contaminants.

The carbonaceous material is deposited on ceramic based material (Step 102). The carbonaceous material is deposited on the ceramic material by heating the ceramic material in a solution of carbon precursors to get a uniformly coated layer of carbonaceous material. The ceramic material is heated in a solution of carbon precursor at a temperature ranging from 150-250° C.

The photo catalytic material (oxides of Ti, Zn, Sn and the like) are deposited on ceramic based material coated with carbonaceous material (Step 104). The photocatalytic material is synthesized by the dropwise addition of the precursor into a mixed solvent in an appropriate quantity under continuous stirring. Hydrolysis of the precursor is carried out using a mixture of water and isopropanol which is added in a dropwise manner A thick sol-gel is formed indicating the formation of metal hydroxides. The mixture is allowed to be mixed properly in a magnetic stirrer. The pH of the solution is maintained by the addition of an acid. Now with the addition of ceramic material into the mixture, the deposition process of metal oxides of Titanium (Ti), Zinc (Zn), Tin (Sn) and other active metal oxide nanoparticles is initiated.

The ceramic based material coated with carbonaceous catalytic material is carbonized/phase transformed in inert atmospheric condition for transforming hydroxide to oxides of Ti/Zn/Sn (Step 106). In this step, first the mixture prepared is subjected to slow heating to undergo phase transformation from a gel phase to a dry phase. The mixture is then annealed at a very high temperature with a heating rate of 1-10° C./min up to a temperature of 850° C. in tubular furnace in the presence of inert atmosphere. Annealing is carried out to achieve the carbonization of carbonaceous material and simultaneously the photocatalytic material undergoes phase transformation from its hydroxide form to its oxide form.

The ceramic based material coated with carbonaceous and photocatalytic material is activated by ultraviolet and acid/base treatment (Step 108).

The ceramic based material coated with carbonaceous material deposited with photo catalytic material is washed and neutralized and packed in a plastic frame (Step 110).

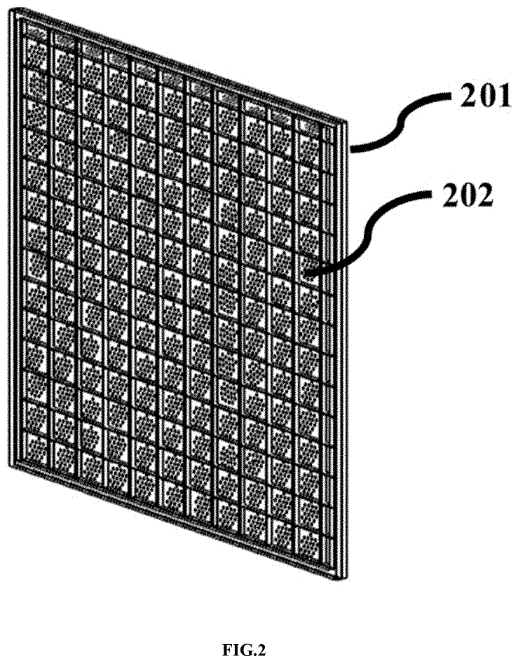

FIG. 2 is a line diagram illustrating isometric line drawing of attachable-detachable type of air filter bed material, according to one embodiment herein. The air filter bed comprises of bed frame 201 and active materials 202. The frame 201 of the air filter bed is made up of very light weight plastic material. The design of air filter frame 201 varies according to the application or requirements. As shown in the FIG. 2, 201 indicates the filter bed frame which holds the active material in its blocks. The frame 201 provides an efficient air flow through the active material placed inside its blocks. FIG. 2 illustrates active materials 202 which are filled in the blocks of the air filter bed. The active material composed of the photocatalytic material like metal oxides, for example Tin, Titanium and Zinc helps in the catalytic degradation of pollutant gases in air like NOx and SOx. Also, the active material that has carbonaceous materials incorporated in it helps in the adsorption of volatile organic compounds (VOCs), deodorization of the air etc.

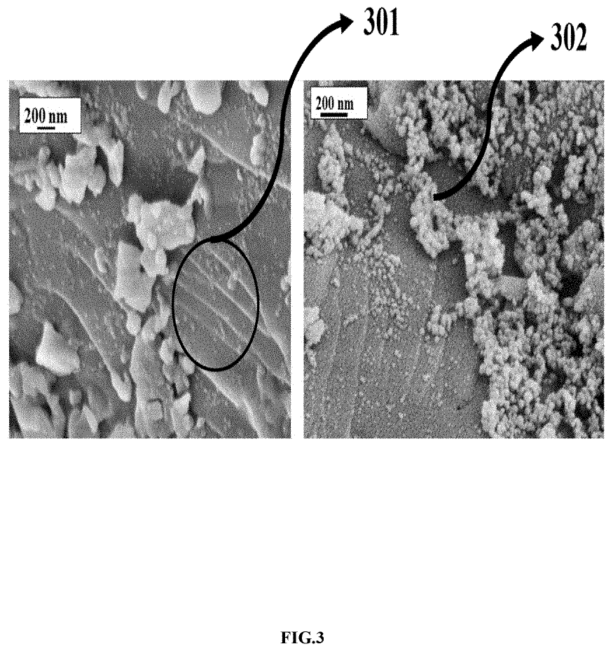

FIG. 3 is a Scanning Electron Microscopy (SEM) images illustrating photocatalytic material coated on the surface of graphene supported ceramic material/ceramic substrate, according to one embodiment herein. The SEM image illustrates graphene sheets denoted by 301 and active metal oxide nanomaterials 302. The metal oxide nanoparticles are seen scattered throughout the surface. A few layers of graphene sheets are also visible from this image as well. The metal oxide immaterial over base material is responsible for the photocatalytic degradation of pollutant gases like NOx and SOx. The adsorption of VOCs and deodorization is carried out by graphene deposited over the ceramic material.

According to one embodiment herein, a photocatalyst supported by graphene-ceramic composite based material filter bed is provided. The photocatalyst supported by graphene-ceramic composite based nano filter comprises photocatalyst supported by graphene ceramic composite in granular or sintered form for removal of various pollutants such as NOx, SOx, VOCs, HCHO and bad odor present in the air.

The graphene illustrates effective antimicrobial and antibacterial property along with dehumidification. The graphene supported photocatalyst nanomaterial is synthesized starting with a ceramic reinforcing material such as silica sand, alumina, zirconia sand or other metal oxide ceramics in particulate form. The ceramic material is first sieved/segregated in desired size and preprocessed by washing with deionized water and acid properly and then dried by heating at elevated temperature. This decontaminates the ceramic materials and activates surface of the ceramic particles.

Following this the ceramic particles are coated with carbon precursor such as sugar, asphalt, tar etc. using a suitable solvent such as water, ethanol, hexane, etc. to get a uniformly coated layer of carbonaceous material over ceramic materials at a temperature ranging from 150-250° C.

The catalytic materials are deposited over carbonaceous material coated ceramic particles. In this step, the photocatalytic materials such as metal oxides of Titanium (Ti) Zinc (Zn), Tin (Sn) and other photocatalytic nanomaterials are synthesized by the dropwise addition of the precursor into a mixed solvent (isopropanol/ethanol etc.) in an appropriate quantity under continuous stirring. Hydrolysis of the precursor is carried out using a mixture of water and solvent such as ethanol/isopropanol etc., which is added in a dropwise manner. A thick sol-gel is formed indicating the formation of metal hydroxides. The mixture is allowed to be mixed properly in a magnetic stirrer. The pH of the solution is maintained by the addition of an acid. Now with the addition of ceramic material into the mixture, the deposition process of photocatalytic nanoparticles is initiated (Step 3).

Further in carbonization and the phase transformation step, the mixture prepared is subjected to slow heating to undergo phase transformation from a gel phase to a dry phase. The mixture is then annealed at a very high temperature with a heating rate of 1-10° C./min up to a temperature of 850° C. in tubular furnace in the presence of inert atmosphere (Argon, Nitrogen, Hydrogen, etc.). Annealing is carried out to achieve the carbonization of carbonaceous material and simultaneously the photocatalytic material undergoes phase transformation from its hydroxide form to its oxide (Step 4). Following this, Ultraviolet activation and Acid/Base activation of the active materials is done (Step 5).

Final step of graphene supported photocatalyst nanomaterials involves the washing of the materials and neutralization (Step 6). Once the graphene-supported photocatalyst nanomaterials coated over ceramic is ready, it is filled in the plastic frame that acts as attachable-detachable type filter where the design of filter frame varies according to the application or requirements such as indoor air purifier, air conditioner, outdoor industrial applications etc.

According to one embodiment herein. Table 1 provides a comparison between a currently available air purifier in the market & graphene supported photocatalyst nanomaterials for the reduction of various VOCs, SOx, and NOx when air is circulated through their respective beds for 1 hour and 24 hours.

| | | | REDUCTION PERCENTAGE | | REDUCTION PERCENTAGE | |
|---|---|---|---|---|---|---|
| | | | (After 1 hour of air circulation) | | (After 24 hours of air circulation) | |
| | | | | GRAPHINE | | GRAPHENE |
| | | | | SUPPORTED | | SUPPORTED |
| Sr. | TARGET | TEST | AIR | PHOTOCATALYST | AIR | PHOTOCATALYST |
| No. | ENTITY | METHOD | PURIFIER | NANOMATERIALS | PURIFIER | NANOMATERIALS |
| 1 | Benzene | IS: 5182 | 39.48% | 51.42% | 72.15% | 79.26% |
| | | Part XI | | | | |
| 2 | Formaldehyde | NIOSH | 22.22% | 33.33% | 64.81% | 72.22% |
| | | 1501 | | | | |
| 3 | Xylene | NIOSH | 49.59% | 54.15% | 61.37% | 65.19% |
| | | 1501 | | | | |
| 4 | Toluene | NIOSH | 23.3% | 29.12% | 59.22% | 64.07% |
| | | 1501 | | | | |
| 5 | Sulphur | IS: 5182 | 8.53% | 21.9% | 39.02% | 40.24% |
| | Oxides | Part II | | | | |
| | (as SO2) | | | | | |
| 6 | Oxides of | IS: 5182 | 26.19% | 35.71% | 41.66% | 44.04% |
| | Nitrogen (as | Part VI | | | | |
| | NOx) | | | | | |
| 7 | Hexane | NIOSH | 20.37% | 27.77% | 50% | 70.37% |
| | | 1501 | | | | |

According to one embodiment herein, Table 2 compares the antibacterial & antibacterial activity of a currently available air purifier in the market & graphene supported photocatalyst nanomaterial by measuring the reduction percentage of selective bacteria & fungi on their respective beds.

| | | | | PERCENT |
|---|---|---|---|---|
| | | | | REDUCTION- |
| | | | | GRAPHENE |
| | | | PERCENT | SUPPORTED |
| | | | RE- | PHOTO- |
| | | | DUCTION- | CATALYST |
| Sr | TARGET | TEST | AIR | NANO- |
| No. | BACTERIA | METHOD | PURIFIER | MATERIAL |
| 1 | Pseudomonas | AATCC TEST | 80.9% | 92.10% |
| | aeruginosa | METHOD | | |
| | (MTCC 424) | 100-2012 | | |
| 2 | E. Coli | AATCC TEST | 80.0% | 91.30% |
| | (MTCC 443) | METHOD | | |
| | | 100-2012 | | |
| 3 | Aspergillus | AATCC TEST | 76.0% | 87.5% |
| | niger | METHOD | | |
| | (MTCC 282) | 100-2012 | | |

According to one embodiment herein, a graphene based active material filter bed system is used in the domestic and industrial applications for removing harmful toxic components present in air.

According to one embodiment herein, an active filter bed system for purification of air is provided to include photocatalytic material for catalytic degradation of gaseous and volatile pollutants, and wherein the photocatalytic material is coated strongly on a ceramic base.

According to one embodiment herein, an Ultraviolet Light Source is provided to activate the photocatalytic material of the bed.

According to one embodiment herein, an Ultraviolet Light Source is provided to also act as a germicidal eliminator and effectively removes microorganisms like bacteria, viruses, yeasts and fungal spores.

According to one embodiment herein, a graphene-based filter for air purification is provided, and wherein the filtration bed comprises active material and wherein the active material comprises graphene supported metal oxide nanoparticles of Titanium, Zinc or Tin, and the like.

According to one embodiment herein, a nanofiltration system for air purification is provided, and wherein the graphene based active material is strongly coated on ceramics like alumina, silica, magnesia, zirconia, iron oxide, etc.

According to one embodiment herein, an air purifier active material bed is provided, and wherein the active material is either in granular form or sintered ceramic bed or rod shaped.

According to one embodiment herein, a graphene supported nanomaterials-based filter system for air purification is provided, to simultaneously remove major gaseous pollutants such as NOx, SOx and volatile pollutants.

According to one embodiment herein, a graphene supported nanomaterials-based filter bed for air purification is provided, and wherein the graphene is chemically functionalized to remove volatile organic compounds (VOCs) including HCHO, benzene and the like.

According to one embodiment herein, a graphene supported nanomaterial-based filter system for air purification is provided to remove bad odor.

According to one embodiment herein, a graphene supported nanomaterials-based filter system with antimicrobial activity is provided for air purification.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

What is claimed is:

1. A method of synthesizing graphene supported photocatalytic nanomaterials used in air purification, the method comprises steps of:
synthesizing a ceramic substrate from a ceramic material in particulate form, and wherein the ceramic material is selected from a group consisting of silica, alumina, zirconia, and metal oxide;
depositing carbonaceous material on the synthesized ceramic substrate to synthesize ceramic substrate coated with carbonaceous material, and wherein the carbonaceous material is selected from a group consisting of sugar, asphalt, and tar;
depositing at least one photocatalytic nanomaterial on the ceramic substrate coated with carbonaceous material, and wherein the at least one photocatalytic nanomaterial is selected from a group consisting of metal oxides of Titanium (Tn), Tin (Sn), and Zinc (Zn);
transforming a phase of the ceramic substrate coated with carbonaceous photocatalytic nanomaterial in inert atmospheric condition from one phase to another phase; and
activating, the transformed ceramic substrate coated with carbonaceous photocatalytic nanomaterial, when exposed to a photo energy source.

2. The method of claim 1, wherein the step of synthesizing the ceramic substrate comprises:
segregating the ceramic material based on size;
washing the segregated ceramic material with deionized water and acid; and
drying the washed ceramic material by heating at elevated temperature;

3. The method of claim 1, wherein the step of depositing the carbon material on the synthesized ceramic substrate comprises mixing the carbon material with a solvent at a temperature ranging from 150-250° C. to obtain a uniformly coated layer of carbonaceous material over the ceramic substrate, and wherein the solvent is selected from a group consisting of water, ethanol, and hexane.

4. The method of claim 1, wherein the step of depositing at least one photocatalytic nanomaterial on the carbonaceous material coated ceramic substrate comprises:
mixing at least one metal element into a mixture of water and solvent, and wherein the solvent is selected from a group consisting of ethanol and isopropanol;
forming a thick solution gel to indicate a formation of metal hydroxides; and
mixing the ceramic material into the thick solution gel to deposit the at least one photocatalytic nanomaterial on the ceramic substrate coated with carbonaceous material.

5. The method of claim 1, wherein the step of transforming the phase of the ceramic substrate coated with carbonaceous photocatalytic nanomaterial comprises transforming of the at least one photocatalytic nanomaterial from metal hydroxides to oxide form.

6. The method of claim 4, wherein the step of transforming the phase of the ceramic substrate coated with carbonaceous photocatalytic nanomaterial comprises:
transforming a phase of the thick solution gel from gel phase to dry phase under slow heating, and;
annealing the transformed solution gel at a second temperature ranging from a heating rate of 1-10° C./min up to a temperature of 850° C. in tubular furnace in presence of inert atmosphere, and wherein the at least one photocatalytic nanomaterial is transformed from hydroxide form to oxide form.

7. The method of claim 1, wherein the photo energy source is an ultraviolet energy source.

8. The method of claim 1, wherein the at least one photocatalytic nanomaterial has at least one of granular form, sintered ceramic bed form, or rod-shaped form.

9. An air purification system comprising:
detachable air filter bed comprising a plurality of blocks:
bed frame for supporting and holding the plurality of blocks, and wherein each of the plurality of blocks is configured to support and hold graphene supported photocatalytic nanomaterials, and wherein the graphene supported photocatalytic nanomaterials are synthesized by performing the steps of:
synthesizing a ceramic substrate from a ceramic material in particulate form, wherein the ceramic material is selected from a group consisting of silica, alumina, zirconia, and metal oxide;
synthesizing a ceramic substrate from a ceramic material in particulate form, and wherein the ceramic material is selected from a group consisting of silica, alumina, zirconia, and metal oxide;
depositing carbonaceous material on the synthesized ceramic substrate to synthesize ceramic substrate coated with carbonaceous material, and wherein the carbonaceous material is selected from a group consisting of sugar, asphalt;
depositing at least one photocatalytic nanomaterial on the ceramic substrate coated with carbonaceous material, and wherein the at least one photocatalytic nanomaterial is selected from a group consisting of metal oxides of Titanium(Tn), Tin(Sn), and Zinc(Zn);
transforming a phase of the ceramic substrate coated with carbonaceous photocatalytic nanomaterial in inert atmospheric condition from one phase to another phase; and
activating, the transformed ceramic substrate coated with carbonaceous photocatalytic nanomaterial, when exposed to a photo energy source.

10. The air purification system of claim 9, wherein the photo energy source is an ultraviolet energy source.
